(19) United States
(12) Patent Application Publication
(10) Pub. No.: US 2015/0050265 A1
(43) Pub. Date: Feb. 19, 2015

(54) ANTI-MCPIP THERAPIES FOR OCULAR NEOVASCULARIZATION

(71) Applicant: Pappachan E. Kollatukudy, Titusville, FL (US)

(72) Inventor: Pappachan E. Kollatukudy, Titusville, FL (US)

(73) Assignee: University of Central Florida Research Foundation, Inc., Orlando, FL (US)

(21) Appl. No.: 14/463,227

(22) Filed: Aug. 19, 2014

Related U.S. Application Data
(60) Provisional application No. 61/867,387, filed on Aug. 19, 2013.

(51) Int. Cl. C07K 16/18; C12N 15/113

(52) U.S. Cl. CPC C07K 16/18; C12N 15/113; A61K 2039/505
USPC 424/130.1; 514/44 A; 514/44 R

(57) ABSTRACT

There is provided a method of treating a condition in a subject in need characterized at least in part by abnormal, uncontrolled, undesirable or pathological ocular neovascularization. The method includes administering to the patient an effective amount of an interfering agent that inhibits the expression of MCPIP.

### FIELD OF THE INVENTION

The present invention relates to methods and compositions for inhibiting ocular neovascularization via knockdown of MCPIP expression. The methods and compositions described herein are useful in preventing and/or treating disorders characterized by abnormal, uncontrolled, undesirable or pathological neovascularization, such as retinopathies.

### BACKGROUND OF THE INVENTION

Undesirable blood vessel growth, e.g., neovascularization, can be observed in many pathologic conditions, including retinopathies. Neovascularization refers to the formation of functional microvascular networks with red blood cell perfusion. It differs from angiogenesis in that angiogenesis is mainly characterized by the protrusion and outgrowth of capillary buds and sprouts from pre-existing blood vessels. Of note, neovascularization can be particularly dangerous if and when vessels swell and burst and/or when neovascularization causes structural damage to the eye. Retinal detachment from the lens, for example, will cause total blindness. In fact, diseases characterized at least in part by neovascularization are the most common cause of blindness across age groups. A major cause of blindness in children, for example, is retinopathy of prematurity (ROP). In working age population adults, proliferative diabetic retinopathy (PDR) is responsible for the highest incidence of acquired blindness. In senior individuals over the age of 64, subretinal neovascularization in age-related macular regeneration (AMD) is the leading cause of blindness.

Inflammatory processes may be characterized by the release of mediators from activated neutrophils, macrophages, and other myeloid cells. The release of these mediators appears to play an active role in new blood vessel development; however, their role is not sufficiently understood. The present inventors have been investigating the role of MCPIP (MCP-1-induced protein) in vasculature formation and different physiological processes. MCPIP was initially isolated from human monocytes after stimulation with MCP-1. The nucleotide (SEQ ID NO: 1) and amino acid (SEQ ID NO: 2) sequences of isolated human MCPIP were deposited with GenBank under accession number AY920403 and the nucleotide (SEQ ID NO: 3) and amino acid (SEQ ID NO: 4) sequences of isolated mouse MCPIP were deposited with GenBank under accession number AY920404. The study of the biological relevance of MCPIP is ongoing.

### BRIEF DESCRIPTION OF THE DRAWINGS

FIG. 1 shows that intravitreal administration of MCPIP expression constructs enhanced neovascularization in a murine model.

FIGS. 2A-2B, on the other hand, show that knockdown of MCPIP via intravitreal injection of siRNA specific for MCPIP strongly inhibited the retinal neovascularization in a murine surrogate model of Proliferative Diabetic Retinopathy (PDR).

### DETAILED DESCRIPTION OF THE INVENTION

Aspects of the present invention are based on the inventors' novel discovery that knockdown of MCPIP levels can reduce neovascularization in an eye of a subject. As shown in the Examples below, the present inventors unexpectedly found evidence, for example, that in a surrogate model of Proliferative Diabetic Retinopathy (PDR), hyperoxia-mediated angiogenesis is mediated by MCPIP. Intravitreal injection of MCPIP-GFP highly advanced neovascularization when compared to the injection of saline or a GFP expression vector in the murine model. In addition, knockdown of MCPIP by via intravitreal injection of siRNA specific for MCPIP strongly inhibited the retinal neovascularization in a murine model of PDR. As such, aspects of the present invention are directed to compositions and methods for the treatment of disorders characterized (at least in part) by abnormal, uncontrolled, undesirable or pathological neovascularization in the eye, such as a retinopathy. Aspects of the present invention provide MCPIP interfering agents, e.g., siRNA or shRNA, which will inhibit MCPIP expression in an eye of a subject, such as by binding to MCPIP mRNA.

In one aspect, the compositions and methods described herein may be utilized as an independent therapeutic agent in the treatment and prevention of disorders, such as retinopathies, characterized at least in part by abnormal, uncontrolled, undesirable or pathological neovascularization in the eye. In another embodiment, the compositions and methods described herein may be utilized in conjunction with other therapies utilized in the treatment of such disorders.

In one aspect, there is provided a method of treating a condition in a subject in need thereof, the condition characterized at least in part by abnormal, uncontrolled, undesirable or pathological ocular neovascularization. The method comprises administering to the subject an effective amount of an interfering agent that inhibits the expression of MCPIP. In an embodiment, the condition is a retinopathy, such as retinopathy of prematurity (ROP), proliferative diabetic retinopathy (PDR), hypertensive retinopathy, or neovascularization in age-related macular regeneration (AMD).

In another aspect, there is provided a method of inhibiting ocular neovascularization in a subject in need thereof. The method comprises administering an effective amount of an interfering agent that inhibits the expression of MCPIP in the eye of the subject.

In still another aspect, there is provided an ophthalmic formulation comprising an effective amount of an interfering agent that inhibits the expression of MCPIP and an ophthalmologically acceptable carrier.

### 1.1 DEFINITIONS

As used herein, the use of the word “a” or “an” when used in conjunction with the term “comprising” in the claims and/or the specification may mean “one,” but it is also consistent with the meaning of “one or more,” “at least one,” and “one or more than one.”

As used herein, the terms “administering” or “administration” of an interfering agent or other therapeutic agent to a subject includes any route of introducing or delivering to a subject a composition as described herein to perform its intended function. In an embodiment, the administering or administration is done ophthalmologically or intravitreally. Administering or administration includes self-administration and the administration by another.

As used herein, the terms “co-administered, “co-administering,” or “concurrent administration”, when used, for example with respect to administration of a conjunctive agent along with administration of an interfering agent as described herein refers to administration of the interfering agent and the conjunctive agent such that both can simultaneously achieve a physiological effect. The two agents, however, need not be administered together. In certain embodiments, administration of one agent can precede administration of the other, however, such co-administering typically results in both agents being simultaneously present in the eye of the subject.

As used herein, the terms “condition,” “disease,” or “disorder” refers to any deviation from a normal state in a subject. In certain aspects, the methods and compositions of the present invention are useful in the prevention and treatment of diseases where the degree of ocular neovascularization differs between subjects with disease and subjects not having disease.

As used herein, by the term “effective amount,” “amount effective,” “therapeutic amount,” “therapeutically effective amount,” or the like, it is meant an amount effective at dosages and for periods of time necessary to achieve the desired result. In the case of the co-administration of an interfering agent with a conjunctive agent as described herein, the conjunctive agent, the interfering agent, or the combination of the interfering agent and the conjunctive agent, may supply the effective amount.

As used herein, the term “expression” in the context of a gene or polynucleotide involves the transcription of the gene or polynucleotide into RNA. The term may also, but not necessarily, involve the subsequent translation of the RNA into polypeptide chains and their assembly into proteins.

As used herein, the term “interfering agent” or the like at least includes all molecules, e.g., RNA or RNA-like molecules, which have a direct or indirect influence on gene expression, such as the silencing of a target gene sequence. Examples of other interfering RNA molecules include siRNAs, short hairpin RNAs (shRNAs), single-stranded siRNAs, microRNAs (miRNAs), methylated siRNAs or other siRNAs treated to protect the siRNA from degradation by circulating RNases, and dicer-substrate 27-mer duplexes. Examples of “RNA-like” molecules include, but are not limited to, siRNA, single-stranded siRNA, microRNA, and shRNA molecules that contain one or more chemically modified nucleotides, one or more non-nucleotides, one or more deoxyribonucleotides, and/or one or more non-phosphodiester linkages. Thus, siRNAs, single-stranded siRNAs, shRNAs, miRNAs, and dicer-substrate 27-mer duplexes are subsets of “interfering agents.” “Interfering agents” also may include ribozymes or PMOs.

As used herein, an “ophthalmologically acceptable carrier” refers to any ophthalmologically acceptable material useful for administering an interfering agent as described herein to an eye of a subject. The carrier preferably does not cause any or a substantial adverse reaction.

As used herein, the terms “phosphothioate morpholino oligomer(s),” “a PMO” or “PMOs” refer to molecules having the same nucleic acid bases naturally found in RNA or DNA (i.e. adenine, cytosine, guanine, uracil or thymine), however, they are bound to morpholine rings instead of the ribose rings used by RNA. They may also be linked through phosphorodiamidate rather than phosphodiester or phosphorothioate groups. This linkage modification eliminates ionization in the usual physiological pH range, so PMOs in organisms or cells are uncharged molecules. The entire backbone of a PMO is made from these modified subunits.

As used herein, the term “antisense compound” refers to an oligomeric compound that is at least partially complementary to a target nucleic acid molecule to which it hybridizes. Antisense compounds include, but are not limited to, compounds that are oligonucleotides, oligonucleosides, oligonucleotide analogs, oligonucleotide mimetics, and chimeric combinations of these.

As used herein, the term “RNA interference” (RNAi) refers to a post-transcriptional gene silencing (PTGS) process whereby one or more exogenous small interfering RNA (siRNA) molecules are used to silence expression of a target gene.

As used herein, “siRNAs” (short interfering RNAs) refer to double-stranded RNA molecules, generally around 15-30 nucleotides in length, that are complementary to the sequence of the mRNA molecule transcribed from a target gene. RNA interference is a two step process. The first step, which is termed as the initiation step, input dsRNA (double stranded RNA) is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 by duplexes (siRNA), each with 2-nucleotide 3′ overhangs [Hutvagner and Zamore Curr Opin Genetics and Development 12:225-232 (2002); and Bernstein, Nature 409:363-366 (2001)]

As used herein, “shRNAs” (small hairpin RNAs) are short “hairpin-turned” RNA sequences that may be used to inhibit or suppress gene expression.

As used herein, a “composition,” “pharmaceutical composition” or “therapeutic agent” all include a composition comprising at least an interfering agent as described herein. Optionally, the “composition,” “pharmaceutical composition” or “therapeutic agent” further comprises one or more pharmaceutically (e.g., ophthalmologically) acceptable diluents, carriers or excipients as described herein. In the case of an interfering agent, for example, the interfering agent may be combined with one or more pharmaceutically acceptable diluents, such as phosphate-buffered saline, for example. As used herein, an ophthalmic composition particularly refers to a composition comprising at least an interfering molecule that is intended to be administered to a subject ophthalmogically or intravitreally as described herein.

As used herein, the term “preventing” means causing the clinical symptoms of the disease state not to develop, e.g., inhibiting the onset of disease, in a subject that may be exposed to or predisposed to the disease state, but does not yet experience or display symptoms of the disease state.

As used herein, the term “subject” or “subject in need” includes any animal (e.g., a mammal), including, but not limited to, humans, non-human primates, rodents, and the like, which is to be the recipient of a particular administration.

As used herein, the terms “treating” or “treatment” or “alleviation” refers to both therapeutic treatment and prophylactic or preventative measures, wherein the objective is to prevent or slow down (lessen) the targeted pathologic condition or disorder.

As used herein, the term “vector” refers to a nucleic acid molecule capable of transporting another nucleic acid to which it has been linked.

### 1.2 INTERFERING AGENT

The interfering agent may be any suitable material capable of inhibiting the expression of MCPIP in vivo or ex vivo. In an embodiment, the interfering agent comprises an interfering molecule that can participate in changes of gene expression of MCPIP, such as inhibiting or silencing expression of MCPIP. Without limitation, for example, the interfering agent may comprise a member from the group consisting of a phosphothioate morpholino oligomer (PMO), microRNA (miRNA), sRNA, methylated sRNA, treated siRNAs, shRNA, antisense RNA, a ribozyme, an antibody, a dicer-substrate 27-mer duplex, and any combination thereof, configured to inhibit the expression of MCPIP to at least an extent.

In a particular embodiment, the interfering molecule comprises a sRNA molecule having a nucleotide sequence specific for at least a portion of the MCPIP sequence. The sRNA molecule may be chemically synthesized, may be produced by in vitro transcription, or may be produced within a host cell. In one embodiment, sRNA is a double stranded RNA (dsRNA) molecule from 15-30 nucleotides in length and may contain a 3′ and/or 5′ overhang on each strand having a length of about 0-5 nucleotides. The length of the overhang is independent between the two strands, e.g., the length of the overhang on one strand is not dependent on the length of the overhang on the second strand. Typically, the sRNA is capable of promoting RNA interference through degradation or specific post-transcriptional gene silencing (PTGS) of the target messenger RNA (mRNA).

It is appreciated that one skilled in the art would readily be able to ascertain a target portion of the MCPIP mRNA material and likewise construct other interfering molecules, such as nucleic acid molecules using a number of techniques known to those of skill in the art. sRNA molecules can be prepared according to the techniques provided in U.S Patent Publication 20060110440, for example, the entirety of which is hereby incorporated by reference.

In another aspect, the interfering agent comprises a shRNA construct. shRNA constructs are typically made from one of three possible methods; (i) annealed complementary oligonucleotides, (ii) promoter based PCR or (iii) primer extension. See Design and cloning strategies for constructing shRNA expression vectors, Glen J McIntyre, Gregory C Fanning, BMC Biotechnology 2006, 6:1 (5 Jan. 2006).

In another aspect, the interfering agent comprises a miRNA molecule. For background information on the preparation of mi RNA molecules, see e.g., U.S. patent applications 20110020816, 20070099196; 20070099193; 20070009915; 20060130176; 20050277139; 20050075492; and 2004/0053411, the disclosures of which are hereby incorporated by reference herein. See also, U.S. Pat. Nos. 7,056,704 and 7,078,196 (preparation of miRNA molecules), incorporated by reference herein. Synthetic miRNAs are described in Vatolin, et al., 2006 J Mol Biol 358, 983-6 and Tsuda, et al 2005 Int J Oncol 27, 1299-306.

### 1.3 DRUG DELIVERY SYSTEMS

The interfering agent may be combined with any suitable delivery system known in the art to improve stability, transport, targeting and efficacy of the interfering agent. Exemplary delivery systems include lipids, peptides, synthetic and natural polymers, viral and non-viral vectors, liposomes, micelles, emulsions, microemulsions, microtubes, and nanotubes. When the interfering molecule comprises a nucleotide sequence, the delivery system may comprise a regulatory sequence useful in expression constructs/vectors with the nucleotide sequence. Exemplary regulatory sequences may include a constitutive promoter, an inducible promoter, a tissue-specific promoter, or a combination thereof.

In a particular embodiment, the delivery system comprises a liposome. Liposomes comprising various lipid compositions useful in delivering an interfering molecule as described herein are known in the art. See Fraley, R., and Papahadjopoulos, D., Trends Biochem. Sci. 6: 77-80). Exemplary liposome preparations are available, such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art.

In another particular embodiment, the delivery of an interfering agent that inhibits MCPIP and thus inhibits neovascularization in an eye of a subject may be accomplished using a number of recombinant DNA and gene therapy technologies, including viral vectors. Preferred viral vectors exhibit low toxicity to the host and produce therapeutic quantities of a molecule that modulates angiogenesis. Viral vector methods and protocols are reviewed in Kay et al., Nature Medicine 7:33-40, 2001. Viral vectors useful in the invention include those derived from Adeno-Associated Virus (AAV). A preferred AAV vector comprises a pair of AAV inverted terminal repeats, which flank at least one cassette containing a promoter which directs expression operably linked to a nucleic acid encoding a molecule that modulates angiogenesis. Methods for use of recombinant AAV vectors are discussed, for example, in Tal, J., J. Biomed. Sci. 7:279-291, 2000 and Monahan and Samulski, Gene Therapy 7:24-30, 2000 and in US Published Patent Application No. 20110091510, the entirety of which is incorporated by reference.

In a particular embodiment, the interfering molecule may be delivered via using a lentivirus. Lentiviruses are a subclass of retroviruses. They have recently been adapted as gene delivery vehicles (vectors) due to their ability to integrate into the genome of non-dividing cells, which is the unique feature of lentiviruses as other retroviruses may only infect dividing cells. The lentivirus may be particularly suitable for in vivo evaluation of an inhibitor. See e.g., Song Y, Zhang Z, Yu X, Yan M, Zhang X, Gu S, et al. (2006). Application of lentivirus-mediated RNAi in studying gene function in mammalian tooth development. Dev Dyn 235:1334-1344.

### 1.4 OPHTHALMIC COMPOSITIONS

In another aspect, the interfering agent and delivery system (if present) may further be formulated as an ophthalmic composition suitable for administration to a subject as defined herein. The ophthalmic composition may be administered ophthalmically or intravitreally using suitable compositions, devices, and methods known in the art. It is appreciated that the administration of the interfering agent-containing compositions described herein may be administered in one dose, multiple doses, continuously or intermittently throughout the course of treatment.

In an embodiment, the ophthalmic composition comprises the interfering agent and an ophthalmically acceptable carrier. In an embodiment, the ophthalmologically acceptable carrier comprises an aqueous solution. In certain embodiments, the ophthalmic formulation is in the form of an eye drop or gel for administration to the eye. In other embodiments, the ophthalmologically acceptable carrier comprises an oil-in-water emulsion or an oil. In such embodiments, the ophthalmic formulation may be in the form of a cream for administration to the eye.

In other embodiments, the carrier may comprise a biodegradable polymer. In certain embodiments, for example, for a biodegradable polymer ocular insert for extended release of the interfering agent may be employed. The ophthalmic carrier may also comprise ophthalmologically acceptable excipients. Excipients suitable for use in the ophthalmic formulation of the present invention include, for example, demulcents, emollients, hypertonicity agents, preservatives, buffers, or pH adjusting agents. Exemplary excipients are described in WO2013091020 A2, for example, the entirety of which is hereby incorporated by reference herein.

In certain embodiments, the ophthalmic formulation may also be used as a vehicle for delivering an additional therapeutic agent to an eye of a patient. Thus, the ophthalmic formulation may comprise a conjuvant agent for treating a retinopathy, for example. The conjuvant agent may, for example, be an anti-inflammatory agent, an anti-immune response agent, an antibiotic, or any other therapeutic agent known to be effective in treating a retinopathy.

When the composition is prepared for intravitreal injection, the compositions may be formulated as described in U.S. Pat. No. 8,211,880, the entirety of which is incorporated by reference herein. In an embodiment, for example, the ophthalmic composition may comprise one or more pharmaceutically acceptable chloride salts, such as tonicity-adjusting agents. Exemplary tonicity-adjusting agents comprise sodium chloride, potassium chloride, calcium chloride, and magnesium chloride. Optionally, the composition may also contain a pH-adjusting agent, such as NaOH or HCl, and/or a pharmaceutically acceptable buffering agent to maintain the pH of the compositions within the range of 6-7.5. Exemplary buffering agents include sodium acetate and sodium citrate.

Further details on techniques for formulation and administration can be found in the latest edition of REMINGTON'S PHARMACEUTICAL SCIENCES (Maack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

### 1.5 COMBINATION WITH OTHER ANTI-NEOVASCULARIZATION THERAPIES

As indicated above, aspects of the present invention provide interfering agents, e.g., si RNA molecules, selected to inhibit MCPIP expression in a subject. In one embodiment, the interfering agent is administered to a subject in conjunction with an additional therapy for treating or preventing neovascularization in an eye of the subject. In one embodiment, the additional therapy comprises one of focal laser treatment, scatter laser treatment, and a vitrectomy. Focal laser treatment (photocoagulation) can stop or slow the leakage of blood and fluid in the eye. In a scatter laser treatment (panretinal photocoagulation), the areas of the retina away from the macula are treated with scattered laser burns. The burns cause the abnormal new blood vessels to shrink and scar. In a vitrectomy, blood from the middle of the eye (vitreous) as well as any scar tissue that's tugging on the retina is removed and replaced with a saline solution. Any of these therapies can be combined with the administration of the interfering agent as described herein.

In another embodiment, the additional therapy is one effective to reduce an amount of neovascularization in an eye of a subject. Exemplary agents include an anti-inflammatory agent such as Kenalog (triamcinolone); a VEGF inhibitor such as Avastin (Bevacizumab) that blocks the effect of VEGF, which causes leakage and new vessel growth; and an angiogenesis inhibitor such as Macugen (pegaptanib sodium) the prevents or inhibits the growth of new blood vessels. Alternatively, any other anti-neovascularization agents or therapies known for use in the treatment or prevention of a ocular neovascularization may be administered along with the interfering molecule. The conjunctive agent may be administered ophthalmically or intravitreally.

### 1.6 APPLICATIONS

The methods and compositions as described herein may be utilized in the treatment and/or prevention of any disorder characterized by abnormal, uncontrolled, undesirable or pathological neovascularization in the eye. It is understood that in some embodiments, the methods and compositions described herein may be applicable to a “subject in need” who has been diagnosed as having or who has been identified as being at risk of having a disorder characterized by abnormal, uncontrolled, undesirable or pathological neovascularization in the eye. Thus, in one embodiment, the compositions described herein may be administered to a “subject in need” having one or more of the following symptoms: blurry vision, double vision, spots in vision, eye pain, eye redness, decreased peripheral vision, and combinations thereof, which may be indicative of increasing ocular neovascularization.

In an embodiment, the compositions may be administered to a subject for the prevention or treatment of a retinopathy. Without limitation, the retinopathy may be associated with hypertension, diabetes mellitus, age-related macular degeneration, trauma, or any other disorder or cause. Thus, the compositions may be administered to a subject for the prevention or treatment of hypertensive retinopathy; diabetic retinopathy; “wet” or neovascular age-related macular degeneration; radiation retinopathy due to exposure to ionizing radiation; solar retinopathy due to direct sunlight exposure; retinopathy associated with sickle cell disease; retinopathy associated with retinal vascular disease such as retinal vein or artery occlusion; trauma, especially to the head; a disease causing Purtscher's retinopathy; and hyperviscosity-related retinopathy as seen in disorders which cause paraproteinemia

### 1.7 ASSESSING GENE SILENCING

The effectiveness of an administration of the interfering agent to a subject can be assessed by directly detecting the presence of the target nucleic in a cell or subject by known methods in the art. For example, the presence of the target nucleic acid can be detected by Southern blot or by a polymerase chain reaction (PCR) technique using primers that specifically amplify nucleotide sequences associated with the nucleic acid. Expression of the target nucleic acids can also be measured using other known methods. For instance, the target mRNA sequence of MCPIP can be detected and quantified using a Northern blot and reverse transcription PCR (RT-PCR). Expression of MCPIP may also be detected by measuring an enzymatic activity or a reporter protein activity.

As noted above, the terms “inhibiting” or “inhibition” decreases the expression or protein activity, or level of a target gene, or protein encoded by the target gene as compared to a situation wherein no interference has been induced. The decrease may be of at least about 10%, about 20%, about 30%, about 40%, about 50%, about 60%, about 70%, about 80%, about 90%, about 95% or about 99% or more as compared to the expression of a target gene or the activity or level of the protein encoded by a target gene which has not been targeted by an interfering agent, such as an siRNA molecule.

### 1.9 EXAMPLES

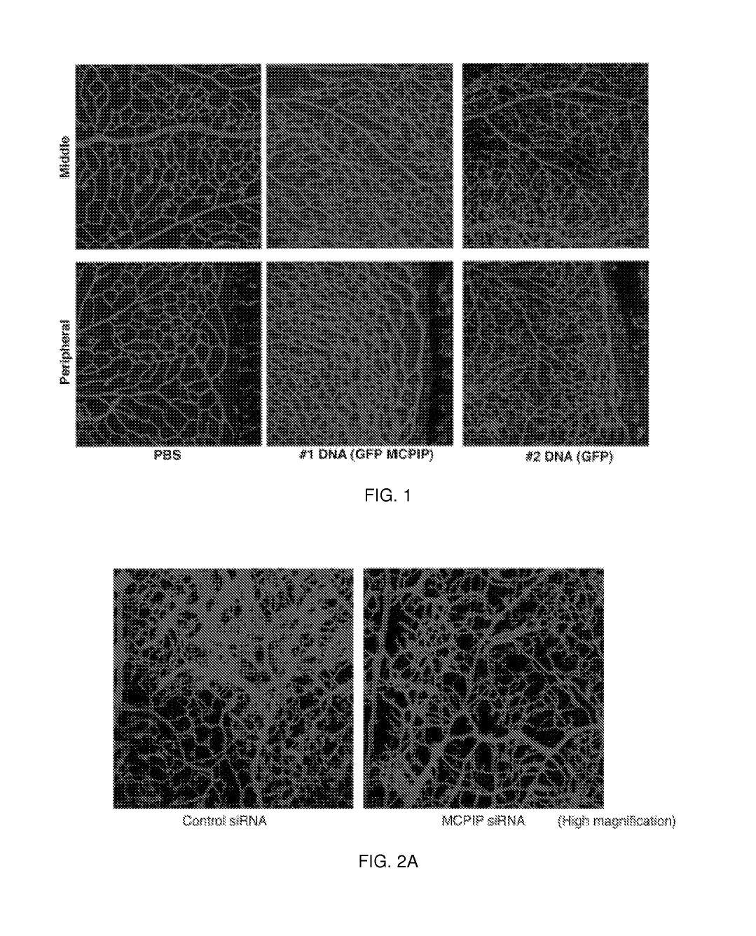

FIG. 1 shows that intravitreal administration of MCPIP expression constructs enhanced neovascularization in a murine model. C57BL mice pups at P8 were used. Expression plasmid for MCPIP-GFP or GFP alone in 10microliter fluid was intravitreally injected at P* and whole mount retina harvested at P10 were examined for vascularization. The intravitreal injection of MCPIP-GFP expression or GFP expression vector was done at P8. The retina samples were harvested for 48 hrs and examined for vascularization.

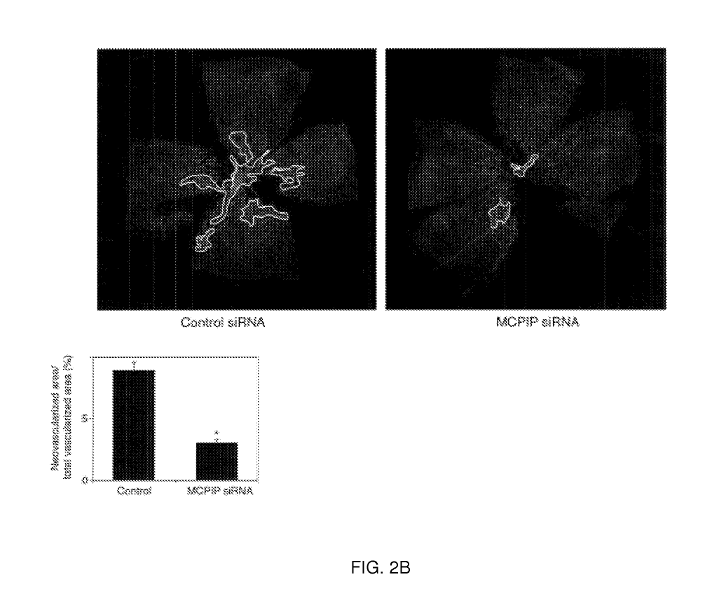

FIG. 2A-2B show that the neovascularization of retina samples were inhibited by intravitreal injection of siRNA specific for MCPIP, but not by scramble siRNA control. After keeping lactating C57BL mice with pups in 75% O2 from P7 to P12 they were transferred to normal air. At this time 10 microliter solution of siRNA specific for MCPIP or scrambled RNA as control was intravitreally injected and the animals were kept in normal room air until P17 when retina were harvested; whole mounted retina were examined for vascularization. Specific siRNA for MCPIP and negative control siRNA were obtained from Ambion. Other siRNAs may be purchased from Santa Cruz Biotechnology (cat no. sc-78944), or Origene (cat nos. Sr312813). shRNA targeting MCPIP is available from Origene (cat no. TF300374). A specific example of siRNA targeting MCPIP includes SEQ ID NO. 1 CTTCGTCAATGACAAGTTT.

FIG. 2A showed that whole mount retina of mice subjected to 75% O₂ from P7 to P12 and then to room air from P12 to P17. A high magnification showing the large reduction in neovascularization by siRNA specific for MCPIP when compared to control scrambled RNA. FIG. 2B includes a quantitative analysis of the extent of neovascularization of murine retina at P17 in a murine model illustrating inhibition of neovascularization at P17. Intravitreal injection of siRNA specific for MCPIP at P12. At P12, vascular obliteration was verified in both sets of animals.

### 1.11 REFERENCES

1. Liang J., Wang J., Azfer A., et al. A novel CCCH-zinc finger protein family regulates proinflammatory activation of macrophages. J. Biol. Chem. 2008; 283:6337-6346.
2. Niu J., Azfer A., Zhelyabovska O., et al. Monocyte chemotactic protein (MCP)-1 promotes angiogenesis via a novel transcription factor, MCP-1-induced protein (MCPIP). J. Biol. Chem. 2008; 283:14542-14551.
3. Skalniak L., Mizaalska D., Zarebski A., et al. Regulatory feedback loop between NF-kappaB and MCP-1-induced protein 1 RNase. FEBS J. 2009; 276:5892-5905.
4. Younce C. W., Kolattukudy P. E. MCP-1 causes cardiomyoblast death via autophagy resulting from ER stress caused by oxidative stress generated by inducing a novel zinc-finger protein, MCPIP. Biochem. J. 2010; 426:43-53.
5. Younce C. W., Azfer A., Kolattukudy P. E. MCP-1 (monocyte chemotactic protein-1)-induced protein, a recently identified zinc finger protein, induces adipogenesis in 3T3-L1 pre-adipocytes without peroxisome proliferator-activated receptor gamma. J. Biol. Chem. 2009; 284:27620-27628.
6. Younce C. W., Wang K., Kolattukudy P. E. Hyperglycemia-induced cardiomyocyte death is mediated via MCP-1 production and induction of a novel zinc-finger protein MCPIP. Cardiovasc. Res. 2010; 87:665-674.
7. JAMA. 2007; 298(8):944. doi:10.1001/jama.298.8.944

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989); Methods in Plant Molecular Biology, Maliga et al, Eds., Cold Spring Harbor Laboratory Press, New York (1995); Arabidopsis, Meyerowitz et al, Eds., Cold Spring Harbor Laboratory Press, New York (1994) and the various references cited therein.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all patents and other references cited herein are incorporated herein by reference in their entirety to the extent they are not inconsistent with the teachings herein.

1. A method of treating a condition in a subject in need thereof, the method comprising administering to the patient an effective amount of an interfering agent that inhibits the expression of MCPIP, wherein the condition is characterized at least in part by abnormal, uncontrolled, undesirable or pathological neovascularization within the eye.

2. The method of claim 1, wherein the subject in need is exhibiting a symptom of the condition.

3. The method of claim 1, wherein the symptom comprises a member selected from the group consisting of blurry vision, double vision, spots in vision, eye pain, eye redness, decreased peripheral vision, and combinations thereof.

4. The method of claim 1, wherein the composition comprises an interfering molecule for interfering with expression of MCPIP.

5. The method of claim 1, wherein the interfering molecule is selected from the group consisting of siRNA, shRNA, miRNA, a ribozyme, a dicer-substrate 27-mer duplex, an antisense nucleotide, an antibody, and a PMO.

6. The method of claim 1, wherein the interfering molecule comprises siRNA.

7. The method of claim 1, wherein the composition comprises an antisense nucleotide.

8. The method of claim 1, wherein the composition comprises shRNA.

9. The method of claim 1, wherein the composition comprises an antibody.

10. The method of claim 1, wherein the condition comprises a retinopathy.

11. The method of claim 10, wherein the retinopathy comprises one selected from the group consisting of diabetic retinopathy, hypertensive retinopathy, and a retinopathy associated with wet macular degeneration.

12. The method of claim 1, wherein the condition comprises retinopathy of prematurity.

13. A method of inhibiting ocular neovascularization in a subject in need thereof, the method comprising:
administering an effective amount of a composition that inhibits the expression of MCPIP in an eye of the subject.

14. The method of claim 13, wherein the subject in need is exhibiting a symptom of ocular neovascularization.

15. The method of claim 13, wherein the symptom comprises a member selected from the group consisting of blurry vision, double vision, spots in vision, eye pain, eye redness, decreased peripheral vision, and combinations thereof.

16. The method of claim 13, wherein the interfering molecule is selected from the group consisting of siRNA, shRNA, miRNA, a ribozyme, a dicer-substrate 27-mer duplex, an antisense nucleotide, an antibody, and a PMO.

17. An ophthalmic formulation comprising:
an effective amount of an interfering agent that inhibits the expression of MCPIP; and
an ophthalmologically acceptable carrier.

18. The ophthalmic formulation of claim 17, wherein the interfering agent comprises an interfering molecule.

19. The ophthalmic formulation of claim 17, wherein the interfering molecule is selected from the group consisting of siRNA, shRNA, miRNA, a dicer-substrate 27-mer duplex, an antisense nucleotide, an antibody, and a PMO specific for MCPIP.

20. The ophthalmic formulation of claim 17, wherein the interfering agent comprises an antisense nucleotide specific for MCPIP.

21-23. (canceled)
